(19) United States
(12) Patent Application Publication
(10) Pub. No.: US 2022/0358764 A1
(43) Pub. Date: Nov. 10, 2022

(54) CHANGE DETECTION AND CHARACTERIZATION OF ASSETS

(71) Applicant: Baker Hughes Holdings LLC, Houston, TX (US)

(72) Inventors: Weiwei Qian, Lexington, MA (US); John Hare, Highland, NY (US); Vladimir Shapiro, Newton, MA (US); Taufiq Dhanani, Boston, MA (US); Rick Hunter, Houston, TX (US); Thai Hoang, Houston, TX (US); Ozge Can Whiting, Providence, RI (US)

(21) Appl. No.: 17/711,549

(22) Filed: Apr. 1, 2022

Related U.S. Application Data
(60) Provisional application No. 63/183,686, filed on May 4, 2021.

(51) Int. Cl. G06V 20/52; G06T 17/00; G06T 19/20; G06V 20/64; G06V 20/10

(52) U.S. Cl. CPC G06V 20/52; G06T 17/00; G06T 19/20; G06V 20/64; G06V 20/176; G06T 2219/2004; G06V 20/56

(57) ABSTRACT

A method for determining a change in an asset and/or a characterization of an asset is provided. In an embodiment, the method can include receiving first data characterizing a target site including one or more assets. The method can also include generating a three-dimensional model of the target site based on the first data. The method can further include registering the first data with the three-dimensional model. The method can also include generating at least one three-dimensional projection onto at least one asset of the one or more assets included in the first data. The method can further include determining second data characterizing the at least one asset based on the at least on three-dimensional projection and providing the second data. In some embodiments, the method can be performed by systems or stored as instructions on computer readable mediums described herein.

### RELATED APPLICATION

This application claims priority under 35 U.S.C. § 119(e) to U.S. Provisional Application No. 63/183,686, filed May 4, 2021, the entire contents of which are hereby expressly incorporated by reference herein.

### BACKGROUND

Industrial operations can include monitoring assets to characterize and detect changes in the assets. Assets can include vessels, industrial equipment, and machinery which can be associated with oil and gas production environments. It can be desirable to monitor changes in the assets or configurations of assets to facilitate asset maintenance planning and inspection.

### SUMMARY

In one aspect, a method for determining a change in an asset and/or a characteristic of an asset is provided. In an embodiment, the method can include receiving, by at least one data processor, first data characterizing a target site including one or more assets. The method can also include generating, by the at least one data processor, a three-dimensional model of the target site based on the first data. The method can further include registering, by the at least one data processor, the first data with the three-dimensional model. The method can also include generating, by the at least one data processor, at least one three-dimensional projection onto at least one asset of the one or more assets included in the first data. The method can further include determining, by the at least one data processor, second data characterizing the at least one asset based on the at least on three-dimensional projection. The method can also include providing the second data.

In some variations, one or more features disclosed herein including the following features may optionally be included in any feasible combination. For example, the first data can include two-dimensional image data. The target site can be an oil and gas production environment. The one or more assets can include at least one of a machine, a vehicle, or a vessel storing a fluid. The first data is acquired by a sensing platform including one or more sensors configured to acquire the first data. The sensing platform can be an aerial sensing platform or a ground-based vehicle. The one or more sensors can include at least one of a position sensor, an image sensor, or a gas detection sensor. The image sensor can be an infrared camera or an RGB camera. The first data can be received by an asset monitoring system including the at least one data processor, the asset monitoring system configured to monitor the target site including the one or more assets. The second data includes at least one of a size of the at least one asset, a location of the at least one asset, or a change in a feature of the at least one asset.

In another aspect, a system for determining a change in an asset and/or a characteristic of an asset is provided. In an embodiment, the system can include a sensor platform including at least one sensor. The system can also include a computing device communicably coupled to the sensor platform via a network. The computing device can include at a display, a memory storing computer readable instructions, a communications interface, and at least one data processor configured to execute the computer readable instructions stored in the memory to cause the at least one data processor to perform operations including receiving first data characterizing a target site including one or more assets. The operations can also include generating a three-dimensional model of the target site based on the first data. The operations can further include registering the first data with the three-dimensional model. The operations can also include generating at least one three-dimensional projection onto at least one asset of the one or more assets included in the first data. The operations can further include determining second data characterizing the at least one asset based on the at least on three-dimensional projection and providing the second data via the display.

In some variations, one or more features disclosed herein including the following features may optionally be included in any feasible combination. For example, the first data can include two-dimensional image data. The target site can be an oil and gas production environment. The one or more assets can include at least one of a machine, a vehicle, or a vessel storing a fluid. The first data can be acquired by the at least one sensor included in the sensing platform. The sensing platform can be an aerial sensing platform or a ground-based vehicle. The at least one sensor can include at least one of a position sensor, an image sensor, or a gas detection sensor. The image sensor can be an infrared camera or an RGB camera. The computing device can include an asset monitoring system configured to monitor the target site including the one or more assets. The second data can include at least one of a size of the at least one asset, a location of the at least one asset, or a change in a feature of the at least one asset.

Embodiments of the present disclosure are directed to improved systems and methods for determining changes and/or characterizations of assets with regard to inspection and monitoring procedures using an asset monitoring system in oil and gas production environments. The systems and methods described herein can be used in inspection and monitoring procedures in other environments without limit.

Non-transitory computer program products (i.e., physically embodied computer program products) are also described herein that store instructions, which when executed by one or more data processors of one or more computing systems, causes at least one data processor to perform operations herein. Similarly, computer systems are also described herein that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

### DESCRIPTION OF DRAWINGS

These and other features will be more readily understood from the following detailed description taken in conjunction with the accompanying drawings, in which:

FIG. 1 is a flow diagram illustrating one embodiment of a method for determining a change in an asset and/or a characterization of an asset according to example implementations described herein;

FIG. 2 is a 2D image of a target site containing assets to be monitored;

FIG. 3 is another 2D image of a target site including overlaid asset contours and detected level lines;

FIG. 4 is a diagram of a system for determining a change in an asset and/or a characterization of an asset according to example implementations described herein; and

FIG. 5 is a block diagram of an exemplary computing system in accordance with an illustrative implementation of the system of FIG. 4.

It is noted that the drawings are not necessarily to scale. The drawings are intended to depict only typical aspects of the subject matter disclosed herein, and therefore should not be considered as limiting the scope of the disclosure.

### DETAILED DESCRIPTION

A common technique for monitoring and inspecting industrial assets is for a human inspector to visit the physical location of industrial assets and to conduct a visual inspection manually. Human inspectors may be unfamiliar with the configuration of the assets, which can lengthen the time necessary to perform inspection procedures. Manual inspection is also prone to error. For example, the human inspector may not adequately recognize changes in an asset or collection of assets in order to properly characterize inspection data as anomalous or requiring further inspection and/or on-going monitoring. Additionally, many industrial assets contain and/or process hazardous materials, which can be poisonous, stored under high pressure, and/or high temperature. Manual inspection of these assets can require human inspectors to wear or utilize expensive, specialized personal protective equipment. Thus, the risks to human inspectors, equipment and the environment are higher when asset inspections are performed manually. Accordingly, manual inspection and monitoring of industrial assets can be time-consuming, hazardous, and cost-prohibitive.

Asset data collected via aerial inspection platforms, such as unmanned aerial vehicles (UAV) or ground-based image acquisition systems, can require specialized processing to ascertain changes or characterizations of the assets being monitored. For example, asset image data generated by such platforms and systems can require sorting by location and asset type prior to visual comparison. Such solutions are often limited because they require a large, comprehensive dataset of images and specialized image processing can be necessary to associate the image data to a particular inspection, type of asset, or location of an asset. Comparing newly collected asset image data to prior collected asset image data can be performed using optical flow techniques to align individual pixels automatically without the need for a three-dimensional (3D) baseline. However, such techniques are susceptible to 3D parallax in the generated comparative view or scene and do not work robustly for industrial assets having variable, complex surfaces and high measures of 3D relief.

To address these technical limitations, the system and methods provided herein can utilize two-dimensional (2D) image data instead of 3D image data. 2D images of a target site including liquid-containing assets to be monitored (e.g., tanks containing oil, water, etc.) can be acquired by one or more manned or unmanned aircraft including drones (e.g., UAVs) equipped with a variety of sensors, including but not limited to a position sensors (e.g., a global positioning system), a gas detection sensor, or an image sensor, such as an infrared camera, and/or a visible light (e.g., RGB) camera to collect image data. In some embodiments, the image data can include infrared or RGB image data. Alternatively, images can be acquired with a camera that is mounted at a fixed position, (e.g., mounted to a post), mounted to a ground-based vehicle, held by hand, or to another structure fixed in place or moveable object without limit.

As discussed in detail below, a combination of 3D reasoning, 3D to 2D image projections, and image processing techniques can be used to determine changes and/or characterizations of assets. For example, by analyzing 2D images of the assets acquired after generation of a baseline 3D model created from 2D images acquired before the generation of the baseline 3D model, the location, size, and nature of changes between portions of the newly acquired 2D images can be provided as time-series image data. For example, the characterizations can include the presence or absence of people, vehicles, asset components, asset configurations, and other objects or assets of interest.

As an example, a drone and a sensor kit mounted on the drone can be employed to monitor assets remotely and regularly. Data from sensor kit can be a stream of images viewing the assets from different viewpoints. The asset image data can be on-boarded in an asset monitoring system and more extensive methods of visual data processing can be performed to extract a 3D representation from the acquired 2D image data using photogrammetry techniques in both RGB and IR modality.

After this onboarding step, assets can be monitored via subsequent routine flights as required by the nature of on-going or reactive inspection or monitoring procedures. The generated 3D model can be analyzed to extract a 3D representation for each asset. Routine inspection images can also be registered to this baseline 3D model using photogrammetry techniques, enabling projection of the 3D representation of the assets into subsequent inspection images. These projections in 2D domain, as well as some 2D image processing techniques, can be used to determine changes or characterizations of assets in a given inspection image.

Additionally, it is recognized that there can be are multiple assets positioned side by side at various target sites, and that these adjacent assets can at least partially occlude acquisition of an image of a given asset. Accordingly, in some embodiments, a depth analysis can be performed to remove portions of an asset in a given image that are occluded by adjacent assets or other objects in that viewpoint. In this way, the determination of changes and characterizations of the assets can be performed more accurately with regard to a specific asset.

Industrial sites can include a large number of assets, such as machinery, vehicles, and vessels storing fluids, such as an oil or a gas, for use during oil and gas exploration, production, transmission, refinement, or distribution operations.

The systems and methods described herein can enable inspection of assets in a manner that is significantly safer for workers than traditional contact-based inspection approaches, especially when assets, such as vessels, can contain toxic materials. Furthermore, by eliminating the need for contact with an asset, accidents that compromise asset integrity and incur costs for repair and/or environmental remediation can be avoided. Additionally, in various embodiments, acquisition of asset image data can be performed in real-time (e.g., at the time of acquisition of the 2D images), near real-time (e.g., immediately after acquisition of the 2D images), or the 2D images can be stored and later retrieved for use in determining changes or characterizations of an asset.

Embodiments of the present disclosure describe systems and methods for asset inspection and monitoring to determine changes in assets and/or characterization of assets in an oil and gas production environment. However, it can be understood that embodiments of the disclosure can be employed for inspecting and monitoring changes and characterizations of assets in any industrial or non-industrial environment without limit.

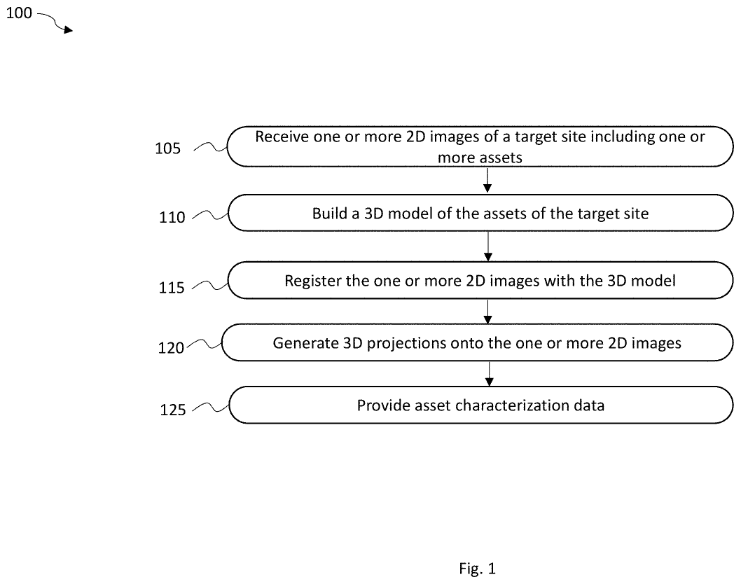

FIG. 1 is a flow diagram illustrating one embodiment of a method 100 for determining a change or a characterization of an industrial asset (e.g., a liquid containing vessel, such as a tank) using an asset monitoring system as described herein. As shown, the method 100 includes operations 105-125. However, it can be understood that, in alternative embodiments, one or more of these operations can be omitted and/or performed in a different order than illustrated. The operations of method 100 described below can apply equally to RGB image data and non-RGB image data.

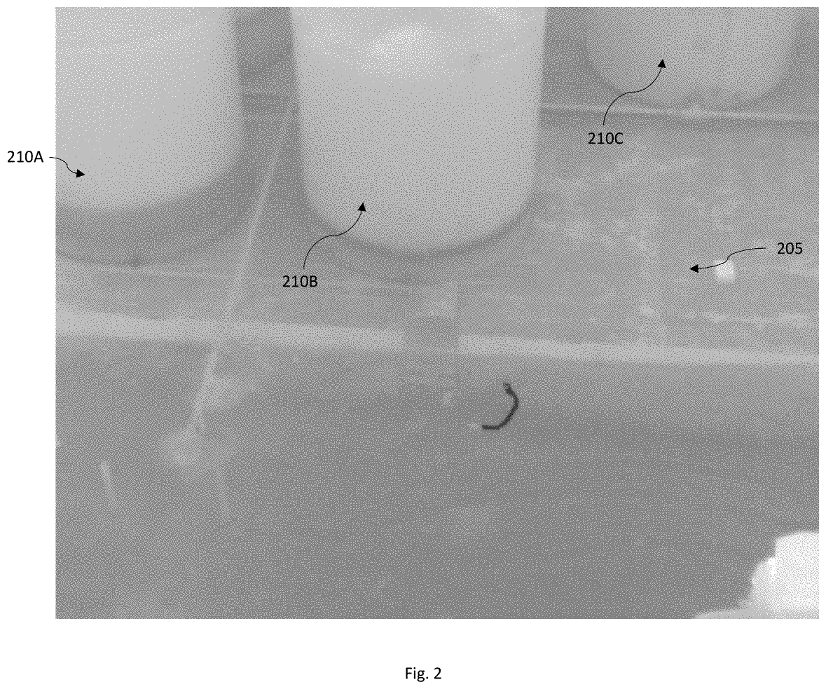

In operation 105, one or more 2D images of a target site including one or more assets can be received by a computing device of an asset monitoring system. For example, as shown in FIG. 2, a target site 205, such as a site targeted for inspection can include one or more assets, such as assets 210A-210C. As an example, the target site 205 can be an oil production environment, and can include assets 210A-210C, such as vessels or tanks containing oil or water.

The 2D images, also referred to as baseline images herein, can be acquired in a variety of ways. In one embodiment, the 2D images can be acquired by at least one image sensor mounted to an aerial vehicle (e.g., a manned airplane, a helicopter, a drone, or other unmanned aerial vehicle). The image sensor can be configured to acquire infrared images, visible images (e.g., grayscale, color, etc.), thermal images, or combination thereof. The image sensor can also be in communication with a position sensor (e.g., a GPS device) configured to output a position, allowing the 2D images to be correlated with the position at which they are acquired. An example of an acquired 2D image of the target site 205 including three assets (e.g., vessels 210A-210C) configured on a well pad is shown in FIG. 2.

Under some circumstances, a given asset can be captured in multiple ones of the second 2D images. Thus, multiple change detections and/or characterizations of the asset can be made. These measurements can be compared to one another and outliers can be eliminated. The remaining measurements can be combined to provide greater accuracy and robustness of the measurement (e.g., averaged). The ability to acquire and/or combine multiple detected changes and/or characterizations of an asset is an important contribution providing robustness and redundancy to the operations described herein.

In operation 110, the 2D images and position information can be analyzed to generate a 3D model of the assets. An example can be found at https://en.wikipedia.org/wiki/3D reconstruction from multiple images. The 3D model can be a baseline 3D model generated as a result of previously acquired 2D image data according to operation 105.

In some embodiments, a new 3D model can be generated from 2D image data at or for a given time ti. The new 3D model can be generated for an entire asset or for a portion thereof. Based on the new 3D model, a 3D change polyhedron can be determined and asset changes can be calculated in 3D as deviations between models at time ti−1 and ti using the 3D change polyhedron. The 3D change polyhedron can be back-projected onto individual 2D images, such as 2D RGB images. By back-projecting the 3D change polyhedron onto individual 2D images, computer vision techniques, such as change area characterizations can be performed on image regions only containing the determined change.

In operation 115, the one or more 2D images can be registered or digitally aligned to the 3D model. For example, newly acquired 2D images of an asset can be collected at times t0, t1, t2, . . . ti. and can be registered to the baseline 3D model by pairing images taken from the same geographic location and using the same camera and/or camera platform orientations with images of the same asset taken from the same geographic location during past acquisitions of the 2D images. In this way, a time series of 2D image data can be defined.

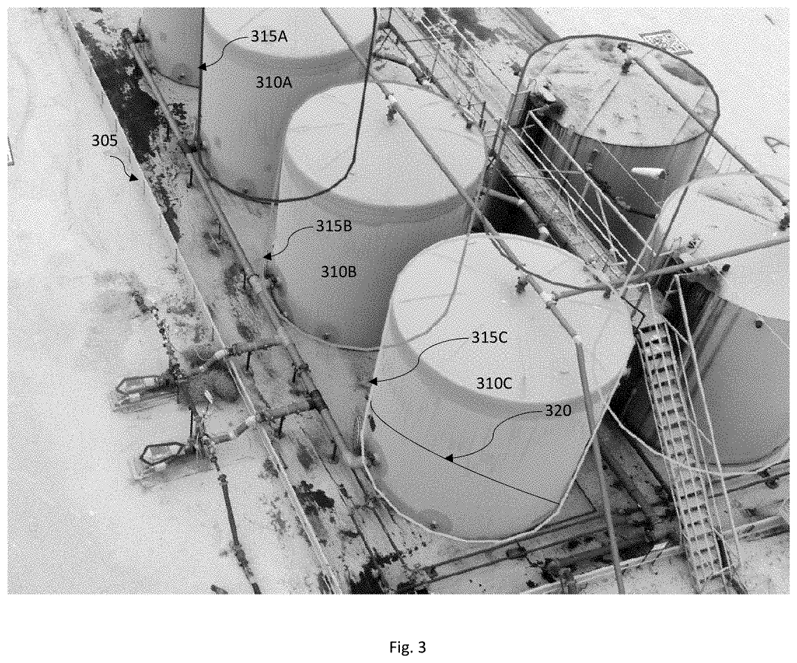

In operation 120, 3D context, such as 3D contours, as shown in FIG. 3, can be projected from the baseline 3D model on each newly acquired 2D image. In this way, a parallax free region of correspondence can be achieved for each 2D image. For example, as shown in FIG. 3, the image of the target site 305 can include assets 310A-310C. 3D contours 315 can be projected onto to the 2D image shown in FIG. 3 and the 3D contours can be provided as contour projections 315 on the corresponding assets 310. Similarly, a fluid level line 320 can also be determined and projected on an asset 310, as shown for asset 310C.

In operation 125, asset characterization data can be provided. The asset characterization data can include a location, size, and nature of change present between corresponding 2D images or regions thereof within the time series data. The asset characterization data can include recognizing the presence and/or absence of people, assets, asset components, configurations of assets, and any other predetermined object of interest, debris, or random things or pieces of equipment left by e.g., technicians. In some embodiments, asset characterization data may be generated for newly acquired 2D images which indicate a change from previously acquired 2D images.

The previously described change and characterization detection operations can also be applied to images collected in different modalities, such as infrared or thermal imaging. For example, an unmanned aerial vehicle (UAV) may be configured with longwave infrared (LWIR) sensors and non-RGB images of assets can be collected. In such embodiments, a 3D model generated from non-RGB image data can be registered or aligned with a baseline 3D model generated from RGB image data using computer vision techniques. As a result, non-RGB images at a time ti can be subsequently aligned to RGB images of the asset to create time series data for the non-RGB images.

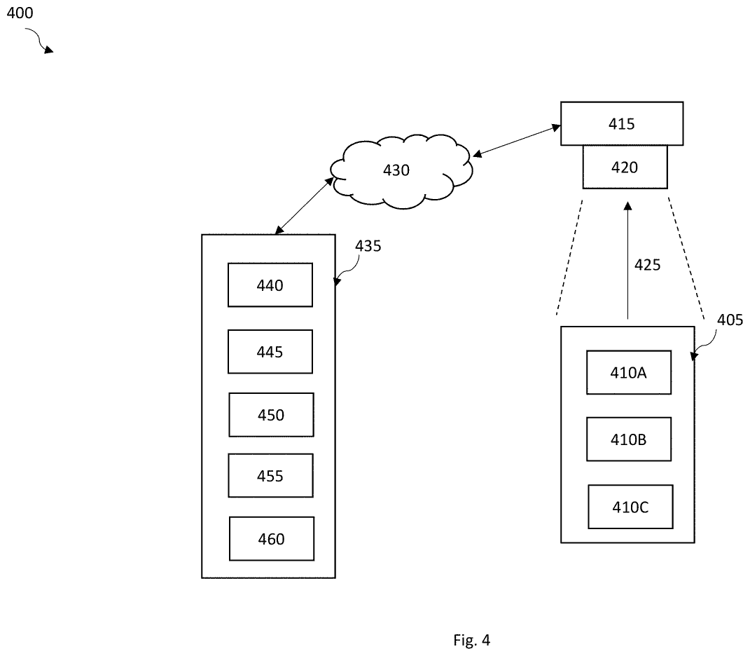

FIG. 4 is a diagram of a system 400 configured to perform the method 100 shown and described in relation to FIGS. 1-3. As shown in FIG. 4, a target site 405 can be the object of an inspection to determine changes in assets or characteristics of assets at the target site 405. The target site can include one or more assets 410, such as 410A-410C. A sensor platform 415, such as a manned or un-manned aerial vehicle, a ground-based vehicle, or a fixed sensing platform, can including at least one sensor 420 configured to acquire sensor data 425 from the assets 410 of the target site 405. The sensor data 425 can vary depending on the type of sensor 420 configured on the sensor platform 415.

The sensor platform 415 can include computerized components, such as a computing device with a processor, memory storing computer readable instructions, and a communications interface that can enable the sensor platform 415 to acquire the sensor data 425 and transmit the sensor data 425 to an asset monitoring system 435 via a network 430. In some embodiments, the sensor platform 415 can receive control signals from the asset monitoring system 435 via the network 430. For example, in regard to determining a change in an asset 410 or a characterization of an asset 410, the asset monitoring system 435 can generate and transmit control signals to the sensor platform 415, that when received and executed by the processor of the sensor platform can cause the sensor platform to change an aspect of its operation, such as adjust an inspection path, or configure a second sensor to collect additional inspection data.

The asset monitoring system 435 can include a number of applications or programs configured to perform aspects of the method and techniques described herein in regard to determining a change in an asset or a characterization of an asset. For example, the asset monitoring system can include an image registration program 440, an asset and 3D model database 445, a 3D model generator 450, a 3D context generator 455, and/or a sensor platform control program 460.

The image registration program 440 can be configured to perform image registration of the 2D image data to the 3D model as described in relation to operation 115 of FIG. 1.

The asset and 3D model database 445 can store data pertaining to the assets 410, such as characteristics or features which may be important to monitor during inspection. For example, the asset and 3D model database 445 can include fluid levels determined for a fluid storage vessel during a prior inspections. The asset and 3D model database 445 can also include 3D models which have been generated and correspond to particular assets 410.

The 3D model generator 450 can include instructions to generate 3D models based on the 2D image data as described in relation to operation 110 of FIG. 1. The 3D model generator 450 can include a variety of geometric and computation model building tools used to generate a 3D model from the 2D image data of the assets 410.

The 3D context generator 455 can include types and attributes of projections to be applied to the characteristics or features of the assets 410 as described in relation to operation 120 of FIG. 1. For example, the 3D context generator 455 can generate and store contour lines associated with particular assets 410 or fluid level lines for particular assets 410. The 3D context generator can include instructions configured to generate the 3D contexts onto the 2D image data.

The sensor platform control program 460 can include instructions configured to control the sensor platform 415 and the sensor 420. The sensor platform control program 460 can generate and cause the asset monitoring system 435 to transmit control signals to the sensor platform 415. The control signals can cause the sensor platform 415 to change a sensor configuration, a flight path, a flight plan, an asset coverage plan, a location, a speed, a direction, a distance to an asset, an inspection protocol or the like. For example, based on an asset characterization determined in regard to operation 125 of FIG. 1, a more vulnerable or important asset can be determined to require additional or more coverage in a monitoring plan of a sensor platform 415. Thus, an aerial sensor platform or ground-based sensor platform may receive control signals to cause the sensor platform 415 to view the asset from different waypoints or different inspection distances.

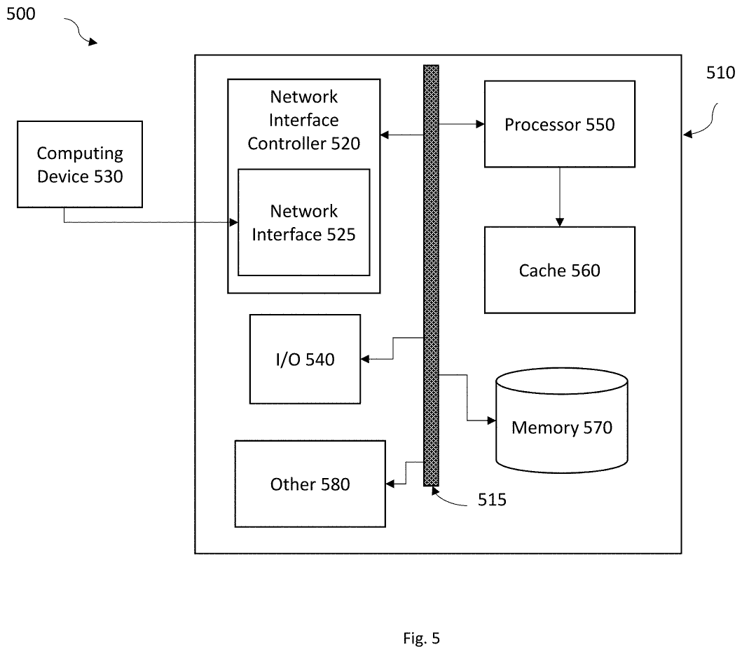

FIG. 5 is a block diagram 500 of a computing system 510 suitable for use in implementing the computerized components described herein, such as one or more computing devices of the sensor platform 415 or the asset monitoring system 435 as shown in FIG. 4. In broad overview, the computing system 510 includes at least one processor 550 for performing actions in accordance with instructions, and one or more memory devices 560 and/or 570 for storing instructions and data. The illustrated example computing system 510 includes one or more processors 550 in communication, via a bus 515, with memory 570 and with at least one network interface controller 520 with a network interface 525 for connecting to external devices 530, e.g., a computing device (such as a computing device 230, or server 255). The one or more processors 550 are also in communication, via the bus 515, with each other and with any I/O devices at one or more I/O interfaces 540, and any other devices 580. The processor 550 illustrated incorporates, or is directly connected to, cache memory 560. Generally, a processor will execute instructions received from memory. In some embodiments, the computing system 510 can be configured within a cloud computing environment, a virtual or containerized computing environment, and/or a web-based microservices environment.

In more detail, the processor 550 can be any logic circuitry that processes instructions, e.g., instructions fetched from the memory 570 or cache 560. In many embodiments, the processor 550 is an embedded processor, a microprocessor unit or special purpose processor. The computing system 510 can be based on any processor, e.g., suitable digital signal processor (DSP), or set of processors, capable of operating as described herein. In some embodiments, the processor 550 can be a single core or multi-core processor. In some embodiments, the processor 550 can be composed of multiple processors.

The memory 570 can be any device suitable for storing computer readable data. The memory 570 can be a device with fixed storage or a device for reading removable storage media. Examples include all forms of non-volatile memory, media and memory devices, semiconductor memory devices (e.g., EPROM, EEPROM, SDRAM, flash memory devices, and all types of solid state memory), magnetic disks, and magneto optical disks. A computing device 510 can have any number of memory devices 570.

The cache memory 560 is generally a form of high-speed computer memory placed in close proximity to the processor 550 for fast read/write times. In some implementations, the cache memory 560 is part of, or on the same chip as, the processor 550.

The network interface controller 520 manages data exchanges via the network interface 525. The network interface controller 520 handles the physical, media access control, and data link layers of the Open Systems Interconnect (OSI) model for network communication. In some implementations, some of the network interface controller's tasks are handled by the processor 550. In some implementations, the network interface controller 520 is part of the processor 550. In some implementations, a computing device 510 has multiple network interface controllers 520. In some implementations, the network interface 525 is a connection point for a physical network link, e.g., an RJ 45 connector. In some implementations, the network interface controller 520 supports wireless network connections and an interface port 525 is a wireless Bluetooth transceiver. Generally, a computing device 510 exchanges data with other network devices 530, such as computing device 530, via physical or wireless links to a network interface 525. In some implementations, the network interface controller 520 implements a network protocol such as LTE, TCP/IP Ethernet, IEEE 802.11, IEEE 802.16, Bluetooth, or the like.

The other computing devices 530 are connected to the computing device 510 via a network interface port 525. The other computing device 530 can be a peer computing device, a network device, a server, or any other computing device with network functionality. For example, a computing device 530 can be a computing device 230 associated with a user of the asset monitoring system 435 or the sensor platform 415 illustrated in FIG. 4. In some embodiments, the computing device 530 can be a network device such as a hub, a bridge, a switch, or a router, connecting the computing device 510 to a data network such as the Internet.

In some uses, the I/O interface 540 supports an input device and/or an output device (not shown). In some uses, the input device and the output device are integrated into the same hardware, e.g., as in a touch screen. In some uses, such as in a server context, there is no I/O interface 540 or the I/O interface 540 is not used. In some uses, additional other components 580 are in communication with the computer system 510, e.g., external devices connected via a universal serial bus (USB).

The other devices 580 can include an I/O interface 540, external serial device ports, and any additional co-processors. For example, a computing system 510 can include an interface (e.g., a universal serial bus (USB) interface, or the like) for connecting input devices (e.g., a keyboard, microphone, mouse, or other pointing device), output devices (e.g., video display, speaker, refreshable Braille terminal, or printer), or additional memory devices (e.g., portable flash drive or external media drive). In some implementations an I/O device is incorporated into the computing system 510, e.g., a touch screen on a tablet device. In some implementations, a computing device 510 includes an additional device 580 such as a co-processor, e.g., a math co-processor that can assist the processor 550 with high precision or complex calculations.

Exemplary technical effects of the methods, systems, and devices described herein include, by way of non-limiting example improved change detection and characterization of industrial assets being inspected and monitored. Changes that can occur on asset surfaces can be more reliably tracked over time providing increased monitoring accuracy, and improved asset maintenance and repair planning. The methods, systems, and devices described herein enable creation of a digital history of asset inspections, changes, and repairs. The methods, systems, and devices described herein enable automated and autonomous data collection and analysis in a more objective manner for a greater percentage of asset inspection coverage as compare to manual inspection and monitoring solutions. As described herein, multimodal asset data can be automatically registered to a location of an asset allowing greater scalability of inspecting multiple assets in different geographical locations on more frequent inspection schedules. Speed of measurement acquisition can be significantly increased compared to conventional, manual inspection by rapid computer-based image analysis as well as analysis of multiple assets substantially simultaneously. By avoiding the need for interaction (e.g., climbing and entry) of human inspectors with monitored assets, the risk of human injury is reduced. Accuracy of image analysis is expected to be high and can be further improved by use of cameras with higher spatial resolution (e.g., RGB cameras).

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

The subject matter described herein can be implemented in analog electronic circuitry, digital electronic circuitry, and/or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The techniques described herein can be implemented using one or more modules. As used herein, the term “module” refers to computing software, firmware, hardware, and/or various combinations thereof. At a minimum, however, modules are not to be interpreted as software that is not implemented on hardware, firmware, or recorded on a non-transitory processor readable recordable storage medium (i.e., modules are not software per se). Indeed “module” is to be interpreted to always include at least some physical, non-transitory hardware such as a part of a processor or computer. Two different modules can share the same physical hardware (e.g., two different modules can use the same processor and network interface). The modules described herein can be combined, integrated, separated, and/or duplicated to support various applications. Also, a function described herein as being performed at a particular module can be performed at one or more other modules and/or by one or more other devices instead of or in addition to the function performed at the particular module. Further, the modules can be implemented across multiple devices and/or other components local or remote to one another. Additionally, the modules can be moved from one device and added to another device, and/or can be included in both devices.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected by any form or medium of digital data communication, e.g., a communication network. Examples of communication networks include a local area network (“LAN”) and a wide area network (“WAN”), e.g., the Internet.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

What is claimed is:

1. A method comprising:
receiving, by at least one data processor, first data characterizing a target site including one or more assets;
generating, by the at least one data processor, a three-dimensional model of the target site based on the first data;
registering, by the at least one data processor, the first data with the three-dimensional model;
generating, by the at least one data processor, at least one three-dimensional projection onto at least one asset of the one or more assets included in the first data;
determining, by the at least one data processor, second data characterizing the at least one asset based on the at least on three-dimensional projection; and
providing the second data.

2. The method of claim 1, wherein the first data includes two-dimensional image data.

3. The method of claim 1, where the target site is an oil and gas production environment.

4. The method of claim 1, wherein the one or more assets include at least one of a machine, a vehicle, or a vessel storing a fluid.

5. The method of claim 1, wherein the first data is acquired by a sensing platform including one or more sensors configured to acquire the first data.

6. The method of claim 5, wherein the sensing platform is an aerial sensing platform or a ground-based vehicle.

7. The method of claim 5, wherein the one or more sensors include at least one of a position sensor, an image sensor, or a gas detection sensor.

8. The method of claim 7, wherein the image sensor is an infrared camera or an RGB camera.

9. The method of claim 1, wherein the first data is received by an asset monitoring system including the at least one data processor, the asset monitoring system configured to monitor the target site including the one or more assets.

10. The method of claim 1, wherein the second data includes at least one of a size of the at least one asset, a location of the at least one asset, or a change in a feature of the at least one asset.

11. A system comprising:
a sensor platform including at least one sensor; and
a computing device communicably coupled to the sensor platform via a network, the computing device including at a display, a memory storing computer readable instructions, a communications interface, and at least one data processor configured to execute the computer readable instructions stored in the memory to cause the at least one data processor to perform operations including
receiving first data characterizing a target site including one or more assets,
generating a three-dimensional model of the target site based on the first data,
registering the first data with the three-dimensional model,
generating at least one three-dimensional projection onto at least one asset of the one or more assets included in the first data,
determining second data characterizing the at least one asset based on the at least on three-dimensional projection, and
providing the second data via the display.

12. The system of claim 11, wherein the first data includes two-dimensional image data.

13. The system of claim 11, where the target site is an oil and gas production environment.

14. The system of claim 11, wherein the one or more assets include at least one of a machine, a vehicle, or a vessel storing a fluid.

15. The system of claim 11, wherein the first data is acquired by the at least one sensor included in the sensing platform.

16. The system of claim 15, wherein the sensing platform is an aerial sensing platform or a ground-based vehicle.

17. The system of claim 15, wherein the at least one sensor includes at least one of a position sensor, an image sensor, or a gas detection sensor.

18. The system of claim 17, wherein the image sensor is an infrared camera or an RGB camera.

19. The system of claim 11, wherein the computing device includes an asset monitoring system configured to monitor the target site including the one or more assets.

20. The system of claim 11, wherein the second data includes at least one of a size of the at least one asset, a location of the at least one asset, or a change in a feature of the at least one asset.
